(19) United States
(12) Patent Application Publication
(10) Pub. No.: US 2024/0125771 A1
(43) Pub. Date: Apr. 18, 2024

(54) REACTION PLATFORM FOR ACCELERATED BIOCHEMICAL REACTION

(71) Applicants: National Taiwan University, Taipei (TW); National Taiwan University Hospital, Taipei (TW); Academia Sinica, Taipei (TW)

(72) Inventors: An-Bang Wang, Taipei (TW); Shih-Yu Chen, Taipei (TW); Tung-Hung Su, Taipei (TW); Chia-Chi Chu, Taipei (TW); Chia-Chien Yen, Taipei (TW); Yu-Wei Chiang, Taipei (TW)

(21) Appl. No.: 18/358,320

(22) Filed: Jul. 25, 2023

Related U.S. Application Data
(60) Provisional application No. 63/392,509, filed on Jul. 27, 2022.

(51) Int. Cl. G01N 33/53; G01N 1/31; G01N 1/34

(52) U.S. Cl. CPC G01N 33/5304; G01N 1/31; G01N 1/34; G01N 2474/20

(57) ABSTRACT

The present invention relates to a reaction platform, which comprises: a machine body with a bottom plate for placing non-porous substrates; and a coater module configured on the top of the machine body and capable of maintaining a preset of a predetermined height for moving along the surface of non-porous substrate, wherein the coater module has one or more slits, and a target liquid can be directly injected or sucking in from the outside of the coater module through the slit, and spreading the target liquid onto a surface of the non-porous substrate while moving along the non-porous substrate; wherein the surface of the non-porous substrate has a target to be coated. The reaction platform of the present invention can not only save time, labor and cost, but also have accurate and reproducible experimental results, showing better results than traditional methods.

### FIELD OF TECHNOLOGY

The present invention relates to a reaction platform, and in particular to a reaction platform that can be used to accelerate biochemical and immunological reaction experiments, but is not limited to this.

### BACKGROUND

Immunohistochemistry (IHC) is a technology that uses antibodies to detect the expression location and expression level of target proteins (specific antigens) in tissue sections, and is often used in biomedical applications, such as pathological diagnosis, exploration of biomarkers, confirmation of target antigens, and development of new drugs.

The procedures of IHC contain multiple steps with several technical tips, which make it not an easy experiment for novice researchers. The tissue handling during the staining procedure is also cumbersome that the specimen needs to be transferred between different washing or staining steps and containers. Many steps in the traditional IHC method are operator-dependent. The staining results are sometimes not reproducible and troubleshooting is frequently needed in case of staining failure. The introduction of automated IHC machines has improved reproducibility and reliability of staining results. However, manual staining method still offers more flexibility in research setting, allowing for optimization of a specific antigen-antibody reaction with better results.

Moreover, the whole staining procedure is time consuming, sometimes it takes hours to a day for the primary antibody incubation. Therefore, it is difficult to staining multiple specimens in a single day manually. In addition, the antibody is costly, and ways to reduce the expense is important in biomedical research, especially in experiments of multiplex IHC when multiple antibodies are used in a single experiment.

### SUMMARY

Considering the above drawbacks, the present invention aims to provide a platform for accelerated and uniform reaction of reactants contained on a non-porous substrate, where the platform includes a coater and a bottom plate, both of which can reduce the amount of reagents and reactants (such as antibodies), accelerate the reaction of reactants, and reduce reaction time through capillary force, thus improving the shortcomings of conventional screening that requires a lot of time and manpower.

An aspect of the present invention provides a reaction platform, comprising: a machine body with a bottom plate for placing a non-porous substrate; and a coater module provided above the machine body and capable of maintaining a preset height for moving along a surface of the non-porous substrate, wherein the coater module has one or more slits, a target liquid can be directly injected or sucked in through the slit from an exterior of the coater module, and the target liquid is coated on a surface of the non-porous substrate when the coater module moves along the non-porous substrate, and wherein the surface of the non-porous substrate has a target to be coated.

In some embodiments, the coater module is connected to a cleaning solution storage tank.

In some embodiments, the coater module has a slit as a first cleaning solution flow channel.

In some embodiments, the bottom plate is connected to a cleaning solution storage tank.

In some embodiments, the bottom plate is provided with a second cleaning solution flow channel.

In some embodiments, the reaction platform further comprises a waste liquid storage tank, and the waste liquid storage tank is connected to the bottom plate for liquid discharge.

In some embodiments, the bottom plate is further provided with an oscillator.

In some embodiments, the reaction platform further comprises a temperature control device provided on one or both sides of the coater.

In some embodiments, the reaction platform further comprises a temperature control device provided around, above, or below the bottom plate.

In some embodiments, the coater module is arranged with multiple non-porous substrates to form one or more slits.

In some embodiments, the reaction platform is used for immunostaining, immunohistochemical staining, immunofluorescent staining, or cell imprint staining.

In some embodiments, the non-porous substrate is a glass slide, plastic, non-metal or metal.

In some embodiments, the target liquid comprises antibodies, molecular probes, drugs, or cells.

Comparing to the conventional techniques, the present invention has the following advantages:

The reaction platform of the present invention can provide a to-be-reacted target contained on a non-porous substrate and a reactant to accelerate and uniform reaction, and the coating head allows manually or electrically driven movement as needed, that can work with bottom plates of different materials, and that can be used together with an additional vibrator to shorten the operation time required.

In addition, the reaction platform of the present invention may further include a temperature control device, where the temperature control device may be provided on the coater or bottom plate, and may accelerate biochemical reactions by increasing the temperature of a target liquid, thereby shortening the operation time of experiments.

The present invention provides a coater module that can be designed to include one or more slits, and that allows a liquid to be distributed evenly to two opposite lateral sides as well as in a downward direction by way of capillary action and gravity, without additional driving power. When the coater module includes a plurality of slits, various reactants can be tested at the same time for their reaction, or lack of reaction, with the to-be-reacted substance adsorbed on a non-porous substrate, thereby increasing detection efficiency significantly.

### BRIEF DESCRIPTION OF THE DRAWINGS

Methods for implementing the techniques of the present invention are described below by way of example, with reference to the accompanying drawings, in which:

FIG. 1 is an external schematic diagram of a reaction platform (1) according to an embodiment of the present invention.

FIG. 2 is an external schematic diagram of a reaction platform (2) according to an embodiment of the present invention.

FIG. 3 is an external schematic diagram of a coater and a bottom plate of a reaction platform according to an embodiment of the present invention.

FIGS. 4A and 4B are oblique side views of a coater of a reaction platform according to an embodiment of the present invention.

FIGS. 5A to 5C are exterior schematic diagrams of a bottom plate according to an embodiment of the present invention.

FIG. 6 is experimental result images of immunohistochemical staining of tonsil tissue slices using different antibodies according to an embodiment of the present invention. Left image are obtained using a traditional staining method; right images are obtained using a reaction platform of the present invention.

FIG. 7 is experimental result images of a sentinel lymph node cell imprint slice obtained using a reaction platform of the present invention according to an embodiment of the present invention.

FIG. 8 is experimental result images of immunofluorescent staining using tonsil tissue according to an embodiment of the present invention. Upper images are obtained using a traditional staining method; lower images are obtained using a reaction platform of the present invention.

### DESCRIPTION OF THE EMBODIMENTS

A detailed description and the technical contents of the present invention are given below with reference to the accompanying drawings. Furthermore, for easier illustrating, the drawings of the present invention are not a certainly the practical proportion and are not limited to the scope of the present invention.

Unless defined otherwise, all technical and scientific terms used herein have the same meaning as commonly understood to one of ordinary skill in the art to which this disclosure belongs. As used throughout the instant application, the following terms shall have the following meanings.

The use of “or” means “and/or” unless stated otherwise. The use of “comprise” means not excluding the presence or addition of one or more other components, steps, operations, or elements to the described components, steps, operations, or elements, respectively. Similarly, “comprise,” “comprises,” “comprising” “include,” “includes,” and “including” are interchangeable and not intended to be limiting. As used herein and in the appended claims, the singular forms “a,” “an,” and “the” include plural referents unless the context dictates otherwise. The terms “a”, “an,” “the,” “one or more,” and “at least one,” for example, can be used interchangeably herein.

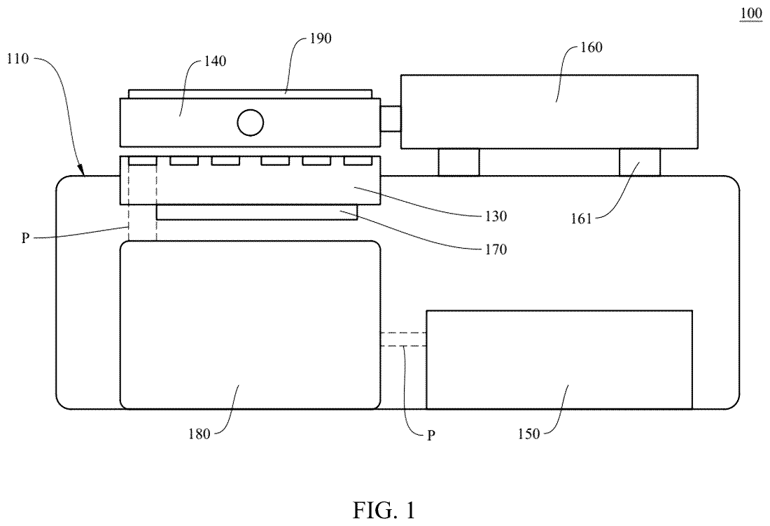

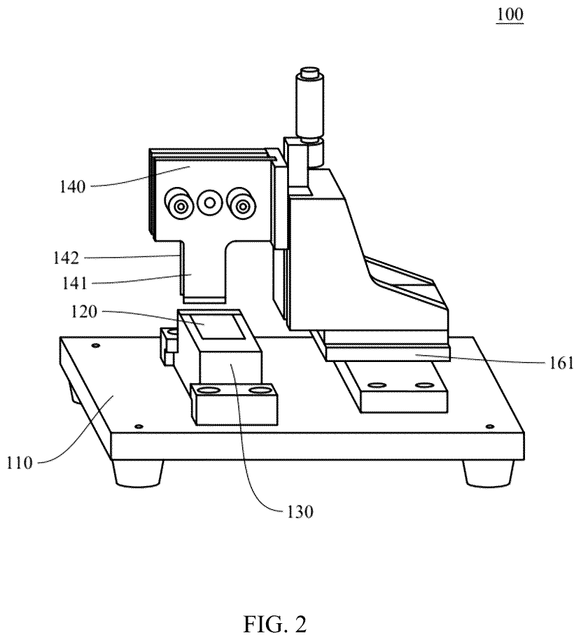

Please refer to FIGS. 1 and 2 together. An implementation aspect of the present invention is a reaction platform 100, which includes a machine body 110, a non-porous substrate 120, a bottom plate 130, and a coater module 140. The machine body 110 has a bottom plate 130 on which the non-porous substrate 120 can be placed; and the coater module 140 is provided above the machine body 110 and can maintain a preset height for moving along a surface of the non-porous substrate 120.

The term “non-porous substrate” herein refers to a substrate material used in biochemical or immune reactions to fix or coat a target substance (or “desired reactant” or “desired coated target”) on it, enabling the “desired reactant” or “desired coated target” to react with a subsequently added target liquid; and compared with a porous substrate, the surface of the non-porous substrate does not have pores and therefore does not absorb liquids. The non-porous substrate includes but is not limited to glass, plastic, non-metal or metal which has a smooth surface; and a material of the plastic may be polyethylene terephthalate (PET), polyvinyl chloride (PVC), polypropylene (PP), or polystyrene (PS). According to at least one embodiment of the present invention, the non-porous substrate is a glass slide or plastic, etc.; in a preferred embodiment, the non-porous substrate is a glass slide.

The term “target liquid” herein may be used interchangeably with “reactant”, which refers to those that can interact with the target substance attached to the non-porous substrate by being coated on the non-porous substrate using a coater (such as but not limited to antigens or antibodies, drugs, pathogens, body fluids, environmental collection fluids, etc.). The term “desired coated target” herein may be used interchangeably with “reactant” or “target substance”, which refers to adsorption or fixation on the surface of the non-porous substrate (such as but not limited to antibodies or antigens, tissues, cells, etc.) for subsequent interaction with its corresponding antigen or antibody or other chemical substance that can interact with it; and the desired coated target may be cells or tissue, such as but not limited to immune cells (i.e. B cells, T cells), liver tissue, or heart tissue.

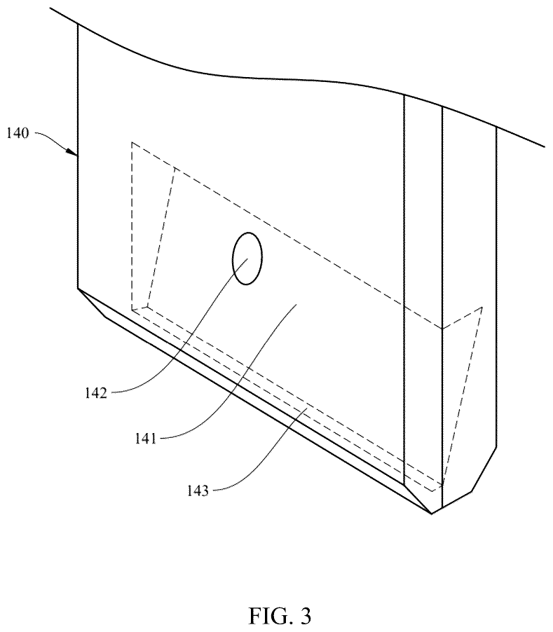

Please refer to FIG. 3. The coater module 140 is arranged with multiple non-porous substrates to form one or more slits 141; one side of the slit 141 is an injection opening 142, while the other side of the slit is a liquid outlet 143, or the injection opening 142 of the slit 141 is also the liquid outlet 143. The injection opening 142 has no special position limit, as long as it can allow the target liquid to flow to the slit 141, so it can also be provided on a side surface of the coater module 140, for example. The liquid outlet 143 is located below the slit 141 of the coater module 140, so that reactants inside the coater module 140 can be uniformly coated on the non-porous substrate.

The term “slit (also known as a hole)” herein refers to a gap formed by a processing method (such as mechanical manufacturing or injection molding) or between two non-porous substrates, and the two non-porous substrates may be parallel or non-parallel, resulting in rectangular, trapezoidal, polygonal, elliptical, circular, and other shapes of the slit; preferably, the two non-porous substrates are parallel; or there are just slits (or holes) in the non-porous substrate to allow the liquid to pass through. The inventor of the present invention has found through experimentation that, as long as the coater module 140 has at least one slit 141 formed between two adjacent non-porous substrates, the injected liquid can be distributed evenly to two opposite lateral sides as well as in a downward direction by way of capillarity and gravity, without additional driving power (e.g., the driving power provided by an injection pump). According to an embodiment of the present invention, the size of the slit 141 (i.e. the point where the slit or hole presents a maximum distance) is 0.1 mm to 1 mm, such as but not limited to 0.1 mm, 0.2 mm, 0.3 mm, 0.4 mm, 0.5 mm, 0.6 mm, 0.7 mm, 0.8 mm, 0.9 mm, or 1 mm. The length of the liquid outlet 143 and the height of the slit 141 can be adjusted according to the size of the coater module, and the present invention does not impose any limitations.

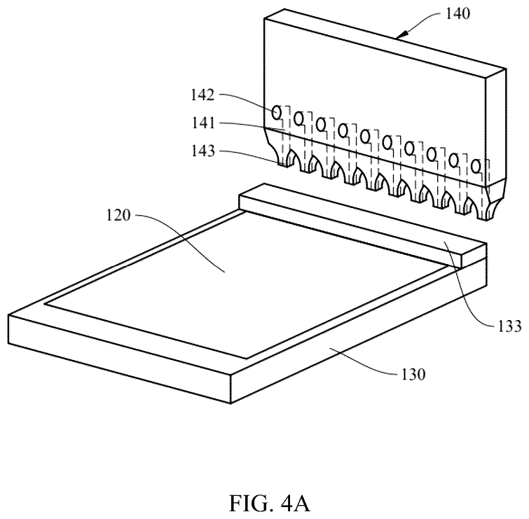

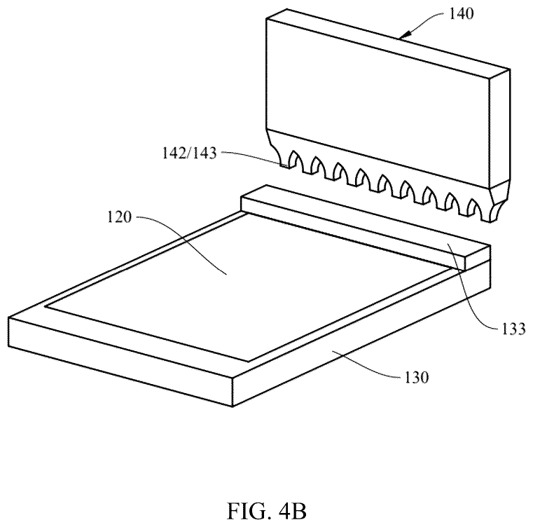

Please refer to FIGS. 3 and 4 together. As shown in FIG. 3, the coater module 140 is a single slit 141; according to an embodiment of the present invention, if the slit 141 is in a trapezoidal shape, the injection opening 142 is set on any non-porous substrate surface of the slit 141 formed by two non-porous substrates, and the set position is not limited to a center, an edge, or any position on the surface, as long as the injection opening 142 can enable the target liquid to flow to the slit 141. In addition, an interior of the coater module 140 may not have a connecting pipeline, and the liquid is directly injected into the slit 141 through the injection opening 142 from an exterior of the coater module 140, and discharged through the liquid outlet 143; and in other words, the injection opening 142 extends from a side of the coater module 140 to a bottom side of the coater module 140 in an open manner, so the liquid may be directly injected into the slit 141 through the opening on the side of the coater module 140, and the liquid is discharged through the opening on the bottom side of the coater module 140 during the coating process. The coater module 140 in FIG. 4A, on the other hand, has a plurality of slits 141. The slits 141 are independent of, and are not in communication with, one another. Each slit 141 has an injection opening 142 at an upper end and a liquid outlet 143 at a lower end. As the slits 141 are not in communication with one another, a different reactant can be injected into each injection opening 142, making it possible to test various reactants in a single test for their reaction, or lack of reaction, with the to-be-reacted substance adsorbed on the non-porous substrate 120, thereby increasing detection efficiency significantly. In addition, the aspect of the coater module 140 in FIG. 4B differs from that in FIG. 4A in that there is a liquid outlet 143 at a lower end of each slit 141, which is also equivalent to the injection opening 142. A user only needs to place the target liquid on the non-porous substrate 120. When the coater module 140 passes through, the injection opening 142 will suck the target liquid into the coater module 140 due to capillarity, and when the coater module 140 continues to move towards the surface of the non-porous substrate 120, the target liquid will be evenly coated onto the non-porous substrate 120 by capillary force and gravity from the injection opening 142 (which is also the liquid outlet 143 at this time) since the target liquid in the injection opening 142 is continuously in contact with the surface of the non-porous substrate 120.

Specifically, the reaction platform of the present invention directly injects or sucks in a target liquid (not shown in the figure) via the slit 141 through the exterior of the coater module 140, and the target liquid is coated on a surface of the non-porous substrate 120 when the coater module moves along the non-porous substrate 120; the surface of the non-porous substrate 120 has a target to be coated (not shown in the figure). When the reaction platform of the present invention is used for coating, a reactant may, as appropriate, be injected into the coater module 140 either manually (e.g., with a metering tool) or electrically (e.g., through an injection pump), such that the reactant enters the slits 141 through the injection openings 142. The reactant automatically forms a liquid bridge between the liquid outlet 143 and the non-porous substrate 120 by way of capillary action, gravity, and inertia. While the coater module 140 is horizontally moved, the liquid bridge of the reactant is laterally extended with a balance between the viscosity, capillarity, gravity, and inertia of the reactant, and coating is carried out as a result. According to at least one embodiment of the present invention, the target liquid comprises antibodies, molecular probes, drugs, or cells.

Please refer to FIG. 1 again. In an embodiment of the present invention, the reaction platform 100 may further include a washing liquid storage tank 160. Specifically, the coater module 140 is connected to the washing liquid storage tank 160 and can be horizontally moved via the slide rails 161 provided under the washing liquid storage tank 160. The driving force of the slide rails 161 may come from an electric motor or manual operation. The invention has no limitation on the location or driving method of the slide rails 161. In addition, the coater module 140 can be horizontally moved in a manually driven or automatic manner. In a preferred embodiment, a relatively simple and flexible way is to manually drive the coater module 140 into horizontal movement, and in that case, the electric motor for effecting horizontal displacement and the corresponding control system can be dispensed with to lower cost and enhance the convenience of operation.

According to at least one embodiment of the present invention, the coater module 140 has a slit as a first cleaning solution flow channel. In detail, the present invention connects the coater module 140 to a cleaning solution storage tank 160, and uses a slit of the coater module 140 as a cleaning solution flow channel, and the cleaning solution of the cleaning solution storage tank 160 can be coated onto the non-porous substrate 120 through the cleaning solution flow channel for cleaning steps, that is, the present invention can directly clean the non-porous porous substrate 120 on the reaction platform 100. In addition, the slit for the flow of the cleaning solution and the slit for the flow of the target liquid are preferably different slits, so that there is no suspicion of residual cleaning solution in the slit for the flow of the target liquid, and there is no problem of antibody residue in the cleaning solution flow channel to ensure the accuracy of the experimental results.

The reaction platform of the present invention can not only provide a cleaning solution through the coater module 140, but also provide a cleaning solution through the bottom plate 130. According to at least one embodiment of the present invention, the bottom plate 130 is connected to a cleaning solution storage tank 160. Specifically, the bottom plate 130 is provided with a second cleaning solution flow channel. By connecting the bottom plate 130 with the cleaning solution storage tank 160, the cleaning solution can be supplied to the bottom plate 130 through the second cleaning solution flow channel, so that the bottom plate is filled with the cleaning solution for cleaning the non-porous substrate 120. In this state, the cleaning solution is provided below the non-porous porous substrate 120 to avoid direct contact between the desired coated target above the non-porous substrate 120 and the cleaning solution provided above and then being washed away.

Please refer to FIG. 2 again. The coater module 140 of the present invention can be manually or automatically coated in parallel directions on the surface of the non-porous substrate 120, and then manually or automatically move the non-porous substrate 120 to a shaking cleaning tank or an ultrasonic cleaning tank to reduce costs and increase operational convenience.

In the reaction platform of the present invention, the bottom plate 130 is a substrate on which the non-porous substrate 120 can be placed. The bottom plate 130 may be a porous or non-porous material. If the bottom plate 130 is a porous material, its material can be such as ceramics, foam, filter paper, or porous fiber, etc. If the bottom plate 130 is a non-porous material, its material can be such as glass, metal, poly(methyl methacrylate) (PMMA, also known as acrylic), a polymer, silicone, plastic, or a combination of the above, etc. In a preferred embodiment, the bottom plate 130 is preferably made of a non-porous material, as the pores of a porous material are prone to residual reactants and cannot be reused after cleaning; and the bottom plate 130 may also be made of suitable materials or detachable modules according to user needs for easy installation and replacement.

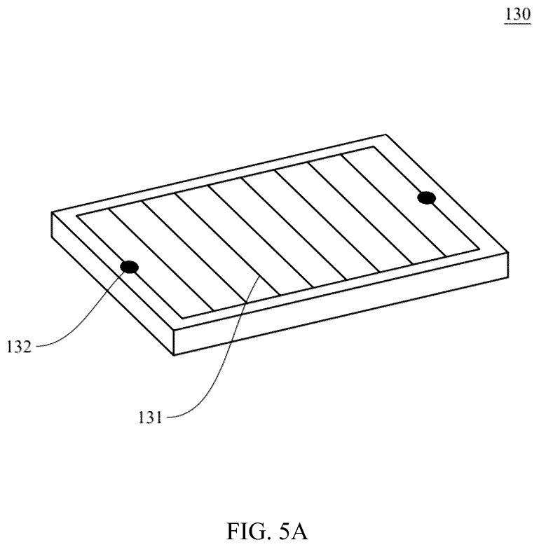

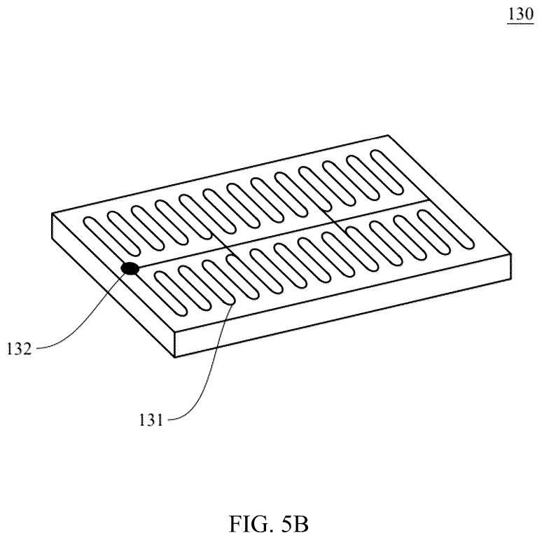

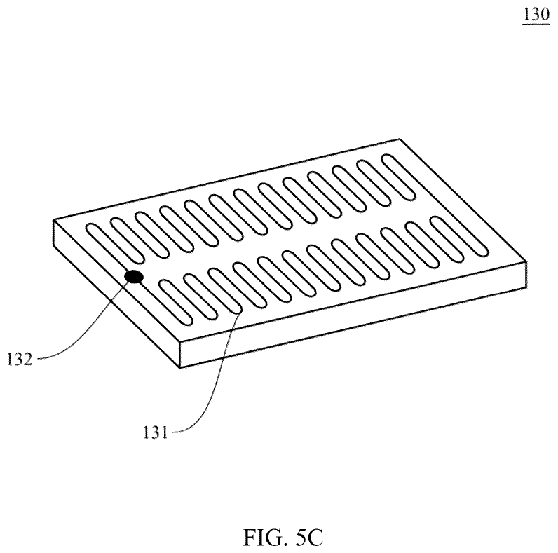

According to at least one embodiment of the present invention, the bottom plate 130 has microchannels 131. The microchannels 131 are thin, tiny structures that contribute to capillarity. Preferably, there is a gradient in the channel depths of the microchannels 131 (i.e., the channel depths gradually increase or decrease), so the liquid in the microchannels 131 will flow through capillarity caused by difference in elevation, with the relatively low channel sections provided at the end where a liquid outlet 132 is located. The liquid outlet 132 is configured for discharging a liquid, and there may be one or more liquid outlets 132. As shown in FIG. 5A, there are two liquid outlets 132 or as shown in FIGS. 5B and 5C, there is only a single liquid outlet 132. In addition, the shape of the microchannels 131 can be as shown in FIG. 5A to 5C, but the present invention does not limit the shape of the microchannels 131. In another embodiment, the liquid outlet 132 is connected to the negative pressure vacuum apparatus 150. Preferably, the liquid outlet 132 is provided in a portion of the microchannels 131 that has a relatively great channel depth. Preferably, coating is carried out by the coater module 140 in a direction from a relatively shallow portion of the microchannels 131 to a relatively deep portion of the microchannels 131. Capillarity together with the depth difference of the microchannels 131 enables the liquid in the microchannels 131 to flow in a predetermined direction (i.e., toward the end where the liquid outlet 132 is located). This flow design not only helps to collect the liquid flowing downwards, but also entrains the reactant containing liquid in the non-porous substrate 120 to be drawn downward.

Besides, the bottom plate 130 may be further provided with a coating preparation zone 133 as shown in FIG. 4 so that the coater module 140 can form a stable liquid bridge in the coating preparation zone 133 before being horizontally moved over the non-porous substrate 120 to carry out the coating process. This allows a preset coating spacing to be maintained, lest the coater module 140 would be contaminated due to its directly contact with the non-porous substrate 120. The coating preparation zone 133 may be integrally formed with or additionally provided on the bottom plate 130.

Another embodiment of the reaction platform of the present invention further includes a negative pressure vacuum apparatus 150, a vibrator 170 and a waste liquid storage tank 180. Referring to FIG. 1, the vibrator 170 is provided under the bottom plate 130 (It can also be installed on the top of the bottom plate 130, not shown in the figure). The waste liquid storage tank 180 is connected to the bottom plate 130 for liquid drainage. In detail, the waste liquid storage tank 180 is provided, and is connected with pipes P, between the bottom plate 130 and the negative pressure vacuum apparatus 150. The washing liquid storage tank 160 is connected to the coater module 140, and the slide rails 161 are provided under the washing liquid storage tank 160. Specifically, the reaction platform according to the foregoing embodiment can be used to wash the non-porous substrate 120. More specifically, when the intended reaction is completed, the non-porous substrate 120 can be coated with the washing liquid from the washing liquid storage tank 160 by the coater module 140, and the microchannels 131 of the bottom plate 130 will keep the non-porous substrate 120 adequately wetted and full of washing liquid. In the meantime, the vibrator 170 will vibrate to wash excess reactant off the non-porous substrate 120. In a conventional washing operation in the laboratory, the non-porous substrate 120 will have to be moved to and soaked in a washing liquid box, allowing spontaneous diffusion to take place under agitation for washing off the excess reactant. This process must be repeated several times to complete the washing and the entire washing operation is labor-intensive and time-consuming. By contrast, washing with the reaction platform of the present invention does not require the movement of the non-porous substrate 120. This reduces the risk of damaging the non-porous substrate 120 while moving it. Also, the vibration-assisted washing process produces a superior washing result to that achievable by natural diffusion and agitation. Furthermore, when the bottom plate 130 is a non-porous material, the microchannels 131 allow the pressure difference between the upstream and downstream ends of the non-porous substrate 120 to be evenly distributed during the liquid suction process so that effective and thorough cleaning can be attained with the smallest possible amount of washing liquid to shorten the operation time required.

In a preferred embodiment, the vibrator 170 is an ultrasonic vibrator, is automatically activated in the washing process for the non-porous substrate 120. Through the laterally back and forth relative motion of tens of thousands of times per second, the enhanced cleaning effect and significantly shorten the washing time can be achieved. The combination of the vibrator 170 and a bottom plate 130 is beneficial not only in washing the non-porous substrate 120 effectively within a short time, but also in suppressing the background noise in the detection signals so as to enhance system performance and signal clarity. Preferably, the embodiment of the present invention uses a bottom plate 130 and an ultrasonic vibrator 170 so that not only can finish effective washing within a short time, but also can effectively suppress the background noise during the signal detection to achieve better system performance and higher signal clarity.

Please refer to FIG. 1 again. According to at least one embodiment of the present invention, the reaction platform 100 further includes a temperature control device 190, and the temperature control device 190 is provided on one or both sides of the coater. In another embodiment, the temperature control device 190 is provided around, above, or below the bottom plate 130. During the reaction process, increasing the temperature of the reaction solution helps to accelerate the reaction; Therefore, the present invention provides a temperature control device 190 for the coater, the position of which is not limited to one or both sides of the coater, as long as the temperature of the coater can be increased, thereby increasing the temperature of the target liquid injected by the coater, and then accelerating the reaction. The temperature control device 190 can also be installed on the bottom plate 130 to increase the temperature of the bottom plate 130, thereby increasing the temperature of the target liquid coated on the bottom plate 130 and accelerating the efficiency of the reaction.

According to at least one embodiment of the present invention, the reaction platform is used for immunostaining, immunohistochemical staining, immunofluorescent staining, or cell imprint staining.

### Embodiment

### Example 1: IHC Staining of Tonsil Tissue Sections with Different Antibodies

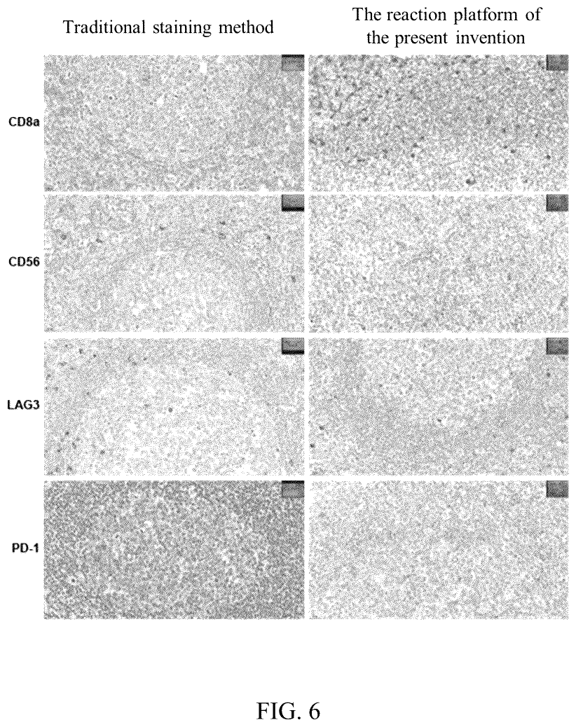

The reaction platform of the present invention is compared with the known IHC method. Referring to FIG. 6, the left side of FIG. 6 is the experimental result of the traditional IHC method, and the right side of FIG. 6 is the experimental result of the reaction platform of the present invention. Tissue sections from human tonsils were used as the positive control sample. We used 5-μm-thick formalin-fixed paraffin-embedded (FFPE) tissue and stained for the T cell membrane marker CD8a. As shown in FIG. 6, CD8a+ cells are largely present in the marginal zone (outside of germinal centers), suggesting the specificity of the staining using the reaction platform.

We further test the staining for additional markers including markers of cell types with low abundance (CD56 for NK cells), markers expressed in the nuclei (LAG3) and markers with weaker intensity (PD-1). All the results from the reaction platform showed the staining patterns consistent with the expected distribution of each marker.

### Example 2: The Imprint Cytology of the Sentinel Lymph Nodes

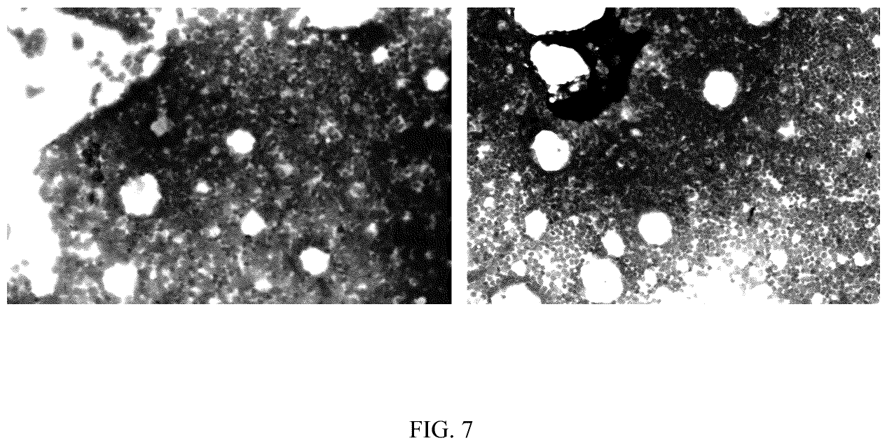

Intraoperational biopsy for the extent of surgical resection is important in the management of several cancers, such as breast cancer. The fresh frozen section or imprint cytology of the sentinel lymph nodes are important to decide the extent of breast surgery, or even a breast conserving surgery. Therefore, the example demonstrates the imprint cytology of the sentinel lymph nodes by the reaction platform of the present invention. Referring to FIG. 7, the results show that the reaction platform can be applied on the staining of imprint cytology, with good results to help the management of diseases.

### Example 3: Immunofluorescence Staining Using Tonsil Tissue

After demonstrating the validity of applying the reaction platform for IHC, we then further evaluate the performance side-by-side between traditional IHC and the reaction platform. The effective staining of IHC only occurs when the antibody collides with the specific target antigen. Therefore, the antibody amount and traveling distance for the antibody to diffuse to the tissue would determine the time required for effective staining. Although higher antibody concentration and longer incubation time would maximize the amount of specific antibody binding to the targeted antigen, at the same time it would also facilitate more non-specific background binding. Given the much shorter traveling distance and total reaction volume for the reaction platform, we reason that the amount of antibody used and the required incubation time would be significantly reduced by using the reaction platform.

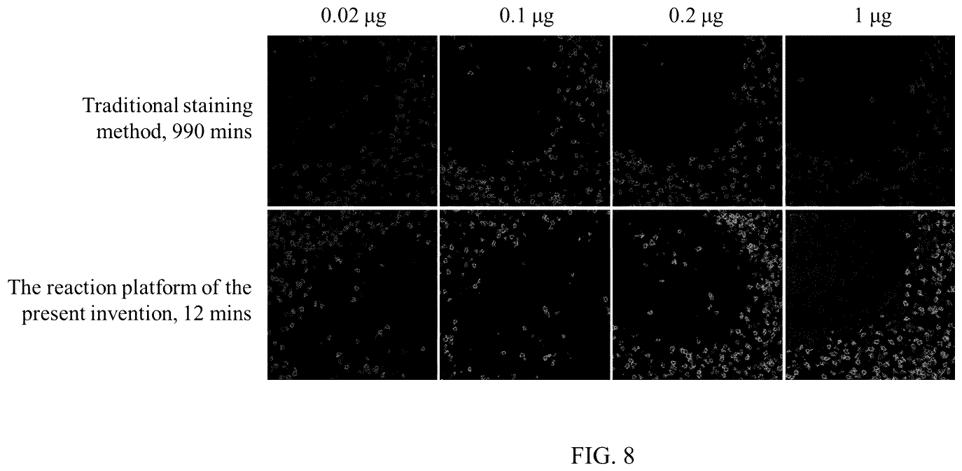

To systematically evaluate the effects of these two factors on the staining performance between traditional IHC and the reaction platform, we conducted a series of experiments by increasing the incubation time to 30 mins and the amount of antibody used from 0.02 μg to 1 μg. In the reaction platform, each tissue section was only coated with a layer of thin-film diluted anti-CD8a antibody solution (from 0.02 to 1 μg/mL with volume of 0.02 mL) measuring some tens of micrometers in thickness and then statically laid on the tissue section for 6 mins. In contrast, the tissue stained with the same amount of antibody for conventional IHC was incubated with a diluted antibody solution (from 0.02 to 1 μg/mL with volume of 0.2 mL) some millimeters-deep for 960 mins. After the incubation with primary anti-CD8a antibody, fluorescent conjugated secondary antibody was applied to the tissue sections (the volume of the secondary antibody in the conventional IHC was 0.2 mL, and the incubation time was 30 mins; the volume of the secondary antibody in the reaction platform of the present invention was 0.02 mL, and the incubation time was 6 mins) followed by imaging with fluorescent microscope for signal intensity. As shown in FIG. 8, the signal intensities across all conditions tested were better in the reaction platform, and it can reduce the amount of antibody and spend less time to achieve a better signal intensities.

In summary, the reaction platform of the present invention allows non-porous substrates to react directly on the platform without the need to transfer the substrates in each step; and through this reaction platform, the consumption of antibodies can be reduced and the time for antibody cultivation can be shortened, in order to save a lot of time and manpower required for experiments. In addition, the reaction platform of the present invention has a standardized experimental process, so the results are repeatable; and compared with traditional immunohistochemical staining methods, it has better effects and exhibits better signal-to-noise ratio.

The above detailed description is a specific description of a feasible embodiment of the present invention, but the embodiment is not intended to limit the scope of the invention. Any equivalent implementation or change that does not depart from the technical spirit of the invention should be included in the scope of the invention.

1. A reaction platform, comprising:
a machine body with a bottom plate for placing a non-porous substrate; and
a coater module provided above the machine body and capable of maintaining a preset height for moving along a surface of the non-porous substrate, wherein the coater module has one or more slits, a target liquid can be directly injected or sucked in through the slit from an exterior of the coater module, and the target liquid is coated on a surface of the non-porous substrate when the coater module moves along the non-porous substrate, and wherein the surface of the non-porous substrate has a target to be coated.

2. The reaction platform of claim 1, wherein the coater module is connected to a cleaning solution storage tank.

3. The reaction platform of claim 2, wherein the coater module has a slit as a first cleaning solution flow channel.

4. The reaction platform of claim 1, wherein the bottom plate is connected to a cleaning solution storage tank.

5. The reaction platform of claim 4, wherein the bottom plate is provided with a second cleaning solution flow channel.

6. The reaction platform of claim 1, further comprising a waste liquid storage tank, and the waste liquid storage tank is connected to the bottom plate for liquid discharge.

7. The reaction platform of claim 1, wherein the bottom plate is further provided with an oscillator.

8. The reaction platform of claim 1, further comprising a temperature control device provided on one or both sides of the coater.

9. The reaction platform of claim 1, further comprising a temperature control device provided around, above, or below the bottom plate.

10. The reaction platform of claim 1, wherein the coater module is arranged with multiple non-porous substrates to form one or more slits.

11. The reaction platform of claim 1, which is used for immunostaining, immunohistochemical staining, immunofluorescent staining, or cell imprint staining.

12. The reaction platform of claim 1, wherein the non-porous substrate is a glass slide, plastic, non-metal or metal.

13. The reaction platform of claim 1, wherein the target liquid comprises antibodies, molecular probes, drugs, or cells.
